(19) United States
(12) Patent Application Publication
(10) Pub. No.: US 2024/0225620 A1
(43) Pub. Date: Jul. 11, 2024

(54) BIOPSY SYSTEM

(71) Applicant: Richard Wolf GmbH, Knittlingen (DE)

(72) Inventors: Bernd Claus WEBER, Karlsruhe (DE); Friedrich HÄHNLE, Bretten (DE); Matthias LAMBERTZ, Bretten (DE); Dirk HÜTTENBERGER, Rammelsbach (DE)

(21) Appl. No.: 18/559,593

(22) PCT Filed: May 10, 2022
(86) PCT No.: PCT/DE2022/200095

§ 371 Date: Nov. 8, 2023

(30) Foreign Application Priority Data
May 14, 2021 (DE) 10 2021 204 906.0

(51) Int. Cl. A61B 10/02; A61B 1/00; A61B 1/04; A61B 1/06; A61B 10/04; A61B 90/00

(52) U.S. Cl. CPC A61B 10/0233; A61B 1/00045; A61B 1/043; A61B 1/0638; A61B 1/0669; A61B 1/0684; A61B 10/04; A61B 90/03; A61B 90/39; A61B 2010/045; A61B 2090/033; A61B 2090/3937

(57) ABSTRACT

A biopsy system includes: a fluorescence endoscopy element (27); a biopsy element (15) for taking a sample (21) of the tissue (3) to be examined from an organic body; and an insertion sleeve (7) for placing a distal end (11, 17) of the fluorescence endoscopy element and/or the biopsy element (15) in or on the tissue (3) to be examined in the organic body. The fluorescence endoscopy element includes a distal section (5) with distal decoupling of stimulation light as well as distal coupling in of fluorescence light. The distal section of the fluorescence endoscopy element is axially moveably positioned relative to the insertion sleeve and can be proximally removed from the insertion sleeve. The biopsy element, when the distal section of the fluorescence endoscopy element is removed, can be placed through the insertion sleeve into or onto the tissue to be examined in the organic body.

### CROSS REFERENCE TO RELATED APPLICATIONS

This application is a United States National Phase Application of International Application PCT/DE2022/200095, filed May 10, 2022, and claims the benefit of priority under 35 U.S.C. § 119 of German Application 10 2021 204 906.0, filed May 14, 2021, the entire contents of which are incorporated herein by reference.

### TECHNICAL FIELD

The present invention relates to a biopsy system for taking tissue samples of an organic body, more particularly for pathological examination.

### BACKGROUND

Often, as part of cancer preventive care or after-care, or in case of suspected cancer, by way of an X-ray, CT or ultrasound examination or using another non-invasive imaging method, an abnormality is diagnosed, which is seen, for example, as a shadow in the examination image. There are various reasons for carrying out such an examination, such as precaution (e.g., screening of the lungs of smokers), follow-up care (e.g. examination of the lungs in the aftercare of colorectal cancer), or an examination for another reason (e.g. investigation of a potentially injured lung after an accident with the incidental finding “round focus”).

Frequently the detection of an abnormality is followed by a treatment (therapy). However, a precondition for a targeted and efficient therapeutic intervention or the decision on whether such a therapeutic intervention is necessary in the first place, is clarifying the precise cause of the abnormality in the examination image. This is because the cause of the shadow can be of completely different types, e.g., tumor, infection, granuloma etc. Different causes sometimes require completely different therapeutic procedures (surgical removal, chemotherapy, radiotherapy, hormonal treatment, pharmacological treatment etc.).

Such clarification (i.e., seeking the cause of the abnormal appearance, i.e. the shadow) takes place by way of an ex situ histopathological or cytological examination of the suspicious tissue. For the pathological examination a piece of tissue from the area of the detected abnormality is needed.

Such a piece of tissue is obtained via tissue removal, known as a biopsy. In principle there are different possibilities for carrying out the tissue removal. If the procedure is to be less burdensome for the patient, the biopsy can, for example, be performed percutaneously (e.g. CT or ultrasonically-controlled in order to make the suspicious area, from where the samples are to be taken, visible). For this, a thin, long and rigid biopsy needle is pushed from outside through the skin and the underlying tissue into the affected organ to the abnormality in order to take a tissue sample there, on or within the abnormality. Usually, hollow needles of different shapes and designs are used in the percutaneous procedure.

Although the images obtained with the usual imaging methods (CT, ultrasound (US), etc.) allow the visualization of suspicious tissue, the degree of tissue differentiation is, however, restricted, i.e. the specificity is limited.

An abnormality as such, does stand out relatively clearly, for example through a different color tone (in CT and US images through a different grey tone) from surrounding healthy tissue. As a result of this it can be comparatively well detected, and the desired removal of tissue carried out comparatively simply in that the distal end of the biopsy needle is guided to the center of the lesion for example.

However, with the usual imaging methods (CT, US etc.), nothing can be said about how or in which way the tissue shown as a shadow has changed, e.g., benignly or malignantly. Thus, for example, in follow-up care, a lesion already treated in the past can still appear as shadowing in CT images, even though this is only fibrously altered, i.e., non-malignant tissue. A biopsy from the center of the shadowing and subsequent pathological examination would confirm this diagnosis.

In the CT or US image, a possibly still present residual tumor (due to, for example, insufficient initial treatment) or even a new tumor formation (relapse, newly occurring after the initial treatment) could not be differentiated from surrounding, fibrously altered tissue shown as shadowing, and would thus remain unnoticed.

A single biopsy from the center of the apparently homogenous abnormality—on the basis of the CT or US there would initially be no reason to take a biopsy at another location—would thus lead to a serious incorrect assessment, as it would not show the non-differentiable residual tumor or new tumor formation in the CT or US image and, accordingly, the actually required therapy would not be carried out.

With the usual imaging methods (CT, US etc.) it can also not be guaranteed that all the suspicious tissue is actually made visible. As well as the specificity, the sensitivity is also limited. This means that an abnormality that is large enough and its tissue is sufficiently greatly altered does stand out relatively clearly as shadowing from the surrounding healthy tissue. In this case, tissue can be taken relatively easily from this area and forwarded for further pathological examination. However, early malignant changes or small malignant changes can often not be shown, or not shown well enough with the usual imaging methods (CT, US etc.). Any additionally present early malignant changes or small malignant changes that lie, for example, in the marginal area of the abnormality or even outside the abnormality, remain invisible in the CT or US image.

A therapy (based on the histopathological or cytological result of the biopsy from the central area of the shadowing) would therefore be restricted to the area of shadowing and a “safety area” around the shadowing. Any early malignant or small malignant changes in the marginal area or outside the shadowing (invisible in the CT or US image), would only be treated inadequately or not treated at all.

In order to resolve this problem, it is known to take a plurality of biopsies from the entire area of the abnormality and also the tissue surrounding the abnormality in order to reduce the risk of overseeing pathologically relevant tissue, that has to be sent for pathological examination. However, a plurality of biopsies has many disadvantages and considerably encumbers the patient. An aggravating fact, i.e. encumbering the patient further, is that a good tissue sample (in the sense of meaningful) should be sufficiently large and therefore the biopsy needle and directly connected thereto also the puncture channel must have a sufficiently large diameter.

In addition, taking many tissue samples is time-consuming, which in addition to placing a burden on the operating person and the treatment costs, also means a further strain on the patient, e.g. long anesthesia. Added to this in CT-supported tissue removal is the increasing radiation load with increasing time. As the tissue samples all have to be histopathologically and cytologically evaluated, such a procedure is also cost-intensive.

### SUMMARY

It is therefore an objective of the present disclosure to provide a biopsy system that, with fewer biopsies, allows a diagnosis to be made more quickly, more cost-effectively and in a manner more gentle on the patient with higher specificity and greater sensitivity.

According to the present disclosure, to solve this task, a biopsy system is provided, wherein the biopsy system comprises
a fluorescence endoscopy element
a biopsy element for taking a sample of tissue to be examined from the organic body, and
an insertion sleeve for placing a distal end of the fluorescence endoscopy element and/or the biopsy element in or on the tissue to be examined in the organic body,
wherein the fluorescence endoscopy element on the proximal side couples in stimulation light and on the distal side decouples the stimulation light as well as on the distal side couples in fluorescence light and on the proximal side decouples the fluorescence light, wherein the distal section of the fluorescence endoscopy element is axially moveably positionable relative to the insertion sleeve and can be removed from the insertion sleeve in the proximal direction,
wherein with the distal section of the fluorescence endoscopy element removed, the biopsy element is placeable through the insertion sleeve into or onto the tissue to be examined in the organic body.

With such a biopsy system, the tissue can already be preselected in situ before actually being taken for the pathological examination. This means that with the known non-invasive imaging methods (CT, US etc.), undifferentiated suspicious-looking tissue can already been evaluated in situ in a more differentiated manner (e.g. with regard to the malignancy), through which the specificity is increased. With the fluorescence endoscopy element, tissue can be stimulated in situ to fluorescence with stimulation light. The pathological tissue stimulated by means of stimulation light, or an accumulation of bacteria indicating pathological tissue can fluoresce and thus be recognizably located relative to the surrounding healthy tissue. The fluorescence endoscopy can be carried out, for example, by means of a photosensitizer or marker substance (e.g., chlorine e6) which selectively accumulates on pathological material and fluoresces. However, it is also possible to dispense with such a photosensitizer and instead the fluorescence of the tissue's own pigments is used (known as autofluorescence), wherein this fluorescence is shown differently depending on the condition of the tissue.

Thus, with the fluorescence endoscopy element, material that is actually pathological can be very precisely localized. When tissue of relevance for the biopsy has been located by way of fluorescence via the fluorescence endoscopy element, the fluorescence endoscopy element can be pulled out of the insertion sleeve, wherein the insertion sleeve remains in the body. The biopsy needle can then be guided through the insertion sleeve precisely to the point where the fluorescence endoscopy element has recognized the tissue to be examined. Therefore, with this biopsy system, more, ideally all relevant, e.g., early malignant and malignant, areas is the organ in question can be localized in situ, without having to take patient-burdening random incidental biopsies from tissue that is not relevant. This means considerably increased sensitivity.

The number of tissue samples that ultimately have to be taken and pathologically examined ex situ is greatly reduced with this biopsy system, which makes diagnosis faster, more gentle on the patient and more cost-effective.

Another advantage of this biopsy system is that the risk is reduced of the taken tissue sample, that is examined pathologically as a relevant piece of tissue and then determines the subsequent therapeutic procedure, is actually taken at precisely the point which was preselected in situ being as relevant (as highly likely to actually be early malignant/malignant). In order words, with this biopsy system the risk of missing the mark with the removal instrument (punch, pincers, biopsy needle etc.) is lower.

Optionally the insertion sleeve can be more rigid than the distal section of the fluorescence endoscopy element. Preferably the distal section of the fluorescence endoscopy element can be configured to be considerably thinner than the biopsy element. It can be advantageous if the insertion sleeve, together with the fluorescence endoscopy element, is for example percutaneously inserted into the body and moved in the direction of the tissue to be examined. The insertion sleeve can then stabilize the distal section of the fluorescence endoscopy element, particularly when piercing solid tissue, e.g., skin and muscles. The insertion sleeve can, but does not have to axially extend into the tissue to be examined. The distal section of the fluorescence endoscopy element can be inserted through softer tissue beyond the distal end of the insertion sleeve into the issue to be examined. The distal section of the fluorescence endoscopy element should at least be so rigid that on puncturing softer tissue it does not bend or only bends a little. In terms of length, the insertion sleeve can be matched to the organ to be examined or an organ-specific insertion sleeve with a defined length for examining a corresponding organ can be selected.

Alternatively, the fluorescence endoscopy element itself can be relatively rigid, for example the distal section of the fluorescence endoscopy element can be guided in a thin-walled tube, which reinforces the distal section of the fluorescence endoscopy element. The process of inserting the distal section of the fluorescence endoscopy element can then initially take place without the help of the insertion sleeve. However, once the final removal position has been found by way of the fluorescence endoscopy element, the insertion sleeve can be pushed from proximal via the distal section of the fluorescence endoscopy element, and inserted distally with the distal section of the fluorescence endoscopy element positioned in situ. As soon as the insertion sleeve is finally positioned in situ, the distal section of the fluorescence endoscopy element is pulled out proximally, wherein the insertion sleeve remains finally positioned in situ. As soon as the distal section of the fluorescence endoscopy element has been completely pulled out of the insertion sleeve, for taking the tissue sample the biopsy element is moved through the insertion sleeve into or onto the tissue to be examined.

Optionally, on the proximal side, the insertion sleeve can be distally everted over the distal section of the fluorescence endoscopy element. This is particularly advantageous if the fluorescence endoscopy element itself is relatively rigid and does not need the insertion sleeve to stabilize the distal section of the fluorescence endoscopy element on insertion into the tissue. Use of the insertion sleeve can thus be held back until the final positioning of the fluorescence endoscopy element has been found. Then, for example, an optical component connected on the proximal side on the distal section of the fluorescence endoscopy element can be uncoupled, and the insertion sleeve, which in this case can be more flexible than the reinforced distal section of the fluorescence endoscopy element, on the proximal side distally everted or pushed over the distal section of the fluorescence endoscopy element. With the already inserted distal section of the fluorescence endoscopy element, the insertion sleeve is then also inserted into the tissue through which the tissue opening widens. This a particularly tissue-sparing procedure, in order to later introduce a thicker biopsy needle into the tissue through the insertion sleeve with the distal section of the fluorescence endoscopy element pulled out.

Optionally, the biopsy element can have a greater radial extent than the distal section of the fluorescence endoscopy element and an inner diameter of the insertion sleeve can be adjustable for optionally receiving the distal section of the fluorescence endoscopy element and the biopsy element. This is particularly advantageous, as the preselection of the tissue by means of the fluorescence endoscopy element is less invasive than inserting the biopsy element. During the preselection it can happen that with the fluorescence endoscopy element no malignant tissue is detected and searching has to take place elsewhere. This spares comparatively patient-burdening tissue removal with the biopsy element at a point that is not relevant. In contrast, puncturing at a point that is not relevant with the thinner distal section of the fluorescence endoscopy element is gentler on the patient.

Optionally, the insertion sleeve can be axially slit and is expandable, preferably over the entire length of the insertion sleeve. The insertion sleeve is preferably largely elastically expandable and made of metal for example. Expanding of the insertion sleeve when introducing the biopsy element is gentler on the patient than directly inserting the biopsy element.

Optionally, for receiving the distal section of the fluorescence endoscopy element, the insertion sleeve is configured in a circumferentially overlapping manner, wherein when the biopsy element is received, the overlapping is smaller or no longer exists. In this way the risk of tissue entrapment can be reduced.

Optionally, the insertion sleeve can comprise an inner sleeve and an outer sleeve, wherein for receiving the biopsy element, the inner sleeve is removable in the proximal direction from the outer sleeve. For receiving the distal section of the fluorescence endoscopy element, the insertion sleeve can thus comprise at least two parts, namely at least one inner sleeve and an outer sleeve, which can be inserted into each other, are detachable from each other and can be displaced with regard to each other.

Optionally, the inner sleeve has an inner diameter that essentially corresponds to an outer diameter of the distal section of the fluorescence endoscopy element, and the outer sleeve has an inner diameter that essentially corresponds to an outer diameter of the biopsy element. To be more accurate, the inner diameter of the inner sleeve is slightly larger than the outer diameter of the distal section of the fluorescence endoscopy element, and the inner diameter of the outer sleeve is slightly larger than the outer diameter of the biopsy element. The inner sleeve may be longer than the outer sleeve. The outer sleeve only has to be inserted so deeply that its position in the tissue is stable. When conspicuous fluorescence is now detected, while retaining the position of the outer sleeve, the distal section of the fluorescence endoscopy element can be pulled out of outer sleeve together with the inner sleeve, and the biopsy element introduced into the outer sleeve and advanced to the site of the conspicuous fluorescence. In the two-part form of embodiment of the insertion sleeve, radial expansion of the insertion sleeve is therefore not necessary.

Optionally, both the fluorescence endoscopy element as well as the biopsy element can have longitudinal markings and/or an axially positionable stop for a defined reproducible relative axial position of the distal section or the biopsy element relative to the insertion sleeve. For example, the fluorescence endoscopy element as well as the biopsy element can each have a length scale, which on the fluorescence endoscopy element shows the distance to the distal tip of the distal section of the fluorescence endoscopy element and on the biopsy element the distance to the tip of the biopsy element. In this way, in a simple manner with the biopsy element the axial position relative to the insertion sleeve (i.e. in the longitudinal direction of the insertion sleeve) which the distal section of the fluorescence endoscopy element previously had can be reproduced. Preferably the longitudinal markings include markings of particular axial positions. For example, the length of the insertion sleeve can be shown in particular, so that the operator can see whether and by how much the distal end of the distal section of the fluorescence endoscopy element and/or the biopsy element is projecting beyond the distal end of the insertion sleeve.

Optionally the insertion sleeve has a flange at a proximal end of the insertion sleeve. The flange can prevent manipulation of the insertion sleeve and full penetration of the insertion sleeve into the skin. On inserting the insertion sleeve, the stop can contact the skin and thus determine the maximum insertion depth of the insertion sleeve. Preferably the length of the insertion sleeve for an organ to be examined, is specially selected so that at maximum insertion depth of the insertion sleeve, wherein the proximal stop of the insertion sleeve is in contact with the skin, the distal end of the insertion sleeve does not penetrate into the tissue to be examined, but ends proximally before it. The actual puncture into the tissue to be examined then only takes place via the distal end of the distal section of the fluorescence endoscopy element projecting beyond the distal end of the insertion sleeve, or with the distal end of the biopsy element projecting beyond the distal end of the insertion sleeve.

Optionally the fluorescence endoscopy element can be configured to optionally proximally couple in a first stimulation light and/or a second stimulation light, wherein the first stimulation light is on average shorter wave (shorter wave length) than the second stimulation light, and wherein the fluorescence endoscopy element is configured to optionally produce a first fluorescence signal from a first tissue area with the first fluorescence light stimulated with the first stimulation light and to receive a second fluorescence signal from a second tissue area from second fluorescence light stimulated with the second stimulation light. As the penetration depth of the shorter wave first stimulation light can be smaller than the penetration depth of the longer-wave second stimulation light, the first tissue area is preferably smaller than the second tissue area. The localization of pathological tissue thus takes place with a higher spatial resolution by way of the first stimulation light. As the penetration depth of the longer-wave second stimulation light can be greater, the detected fluorescence signal can also originate from an correspondingly larger second tissue area. The localization of pathological tissue thus takes place with a lower spatial resolution by way of the second stimulation light, but a larger volume is recorded. Therefore, initially a larger, second, tissue area can be preselected more roughly with the longer-wave second stimulation light, and then a smaller, first tissue area, which is preferably a part of the second tissue area can be examined more closely with the first stimulation light.

Optionally, on the proximal side the fluorescence endoscopy element can have an optical component which is detachably connectable or firmly connected to the distal section of the fluorescence endoscopy element, wherein the optical component has a beam splitter with an image path side and an illumination path side, wherein to decouple the fluorescence light an image sensor or a photodiode or similar is detachably connectable or firmly connected to the image path side, and wherein to couple in the stimulation light, a light source is detachably connectable or firmly connected to the illumination path side. The light source is preferably at least an LED or at least a laser decoupling. The beam splitter can, for example, comprise a pair of prisms which together form a beam splitter block that at least largely separates the stimulation light and the fluorescence light so that the fluorescence light is transmitted or reflected to the image path. Vice versa, the pair of prisms can reflect or transmit the stimulation light to the illumination path side. Preferably the image path side and the illumination path side of the beam splitter lie on sides of the beam splitter arranged at an angle, preferably of around 90° to each other. Preferably the image path side lies on the proximal side, so that fluorescence light is essentially transmitted from the distal input side of the beam splitter to the image path side. The illumination path side is preferably on the lateral side so that stimulation light is essentially reflected from the lateral input towards the distal output side.

At this point it is noted that “image path” is not restricted here to an image being produced from the coupled in fluorescence light. In the image path just a coupled in fluorescence signal can be transmitted, the property of which is determined. Coupling in of fluorescence light by means of an image sensor, a photodiode or similar, does not necessarily have to be used to produce images. For deciding whether the fluorescence signal is conspicuous or not, i.e. whether a biopsy should or should not be taken, it is often enough if only one property of a fluorescence signal, for example its amplitude or intensity, is determined. For example, the conspicuousness can be seen in increased or decreased fluorescence intensity compared to the surrounding tissue or compared to a reference value.

Optionally, the optical component can have an optical longpass filter which transmits the fluorescence light more strongly to the image path side than the stimulation light, and/or comprises an optical shortpass filter which reflects the fluorescence light more strongly to the image path side than the stimulation light. For example, the longpass filter or the shortpass filter can be arranged between a pair of prisms acting as a beam splitter and/or be in the form of a layer on one or both sides of the prisms facing each other. This is particularly sensible if the stimulation light is shorter wave than the fluorescence light.

Optionally, the fluorescence endoscopy element can comprise an operating unit signal-connected or connectable with the image sensor or the photodiode or similar and/or the light source, wherein the operating unit has an optical or acoustic display component, a control and supply component and an operating component.

Optionally, the fluorescence endoscopy element or the optical component can be provided with a handle or a comparable ergonomic component, so that it can also be used as a handling device for introducing the distal section of the fluorescence endoscopy element into the skin and the underlying tissue and therefore such a handling device as a separate and additional unit that has to be removed again from the distal section of the fluorescence endoscopy element after introduction of the light guide into the tissue in order to be able to connect the optical component, can be dispensed with.

Optionally, the biopsy system can also comprise a template, positionable and fixable in relation to the organic body, with at least one opening for the selective receiving of the insertion sleeve and the biopsy element, wherein the insertion sleeve and/or the biopsy element in the at least one opening is fixed against lateral displacement and tilting through the template. This is particularly rational if the skin of the patient at the point of the body to be punctured is not firm enough to fix the insertion sleeve securely in relation to the tissue to be examined. Alternatively or in addition to the skin, the template can be used for fixing the insertion sleeve or the biopsy element.

Such a template can be a flat or three-dimensional structure that is rigid or a least much stiffer than the skin of a patient. The template can, for example, be stuck or strapped to the skin of the patient and thus be fixed relative to the tissue to be examined. Alternatively or additionally, the template can be fixed, for example on an operating table, relative to a reference point. The patient can also be fixed, for example on the operating table, relative to this reference point. The template can be specially matched to an organ to be examined, so that the at least one opening now only allows the operator one degree of freedom of movement for the distal section of the fluorescence endoscopy element or the biopsy element, namely the insertion depth. The lateral positioning and orientation are determined by the at least one opening of the template. The insertion sleeve then no longer needs to be inserted into the body of the patient, but only into the opening, where is acts as a type of adapter to compensate for the difference in thickness between the distal section of the fluorescence endoscopy element and the biopsy element.

Optionally the insertion sleeve can comprise a first part and a second part, wherein the first part fixes the distal section of the fluorescence endoscopy element in the at least one opening against lateral displacement and tipping, and wherein the second part fixes the biopsy element in the at least one opening against lateral displacement and tipping. The two parts can be configured in the form of an inner and outer sleeve inserted into each other or separately each fix the distal section of the fluorescence endoscopy element or the biopsy element. In the case of one-part embodiment of the insertion sleeve, the opening itself can take over the function of the second part and have a corresponding guide surface so that only the first part of the insertion sleeve is needed.

Optionally the template can have a plurality of openings, which are optionally available for receiving the insertion sleeve and the biopsy element. Preferably the template comprises a grid of openings, so that the operator by selecting the opening and by way of the selected insertion depth can seek malignant tissue with the less traumatic fluorescence endoscopy element before the more traumatic biopsy element is used. Several fluorescence endoscopy elements can also be used at the same time. As soon as a point for the biopsy is found through a conspicuous fluorescence signal, the operator only has to reproduce the puncture depth by way of longitudinal markings with the biopsy element. The openings can essentially be orientated in parallel to or at an angle to each other. In the case of larger organs, which may be bigger than the template, a parallel orientation can rational. Particularly in the case of organs that are smaller than the template, for example the prostate, the openings can be aligned at an angle with regard to each other so that they point to the organ from different directions. The template is thus preferably precisely matched to the organ or even individually to separate patients or certain patient groups.

Preferably a grid size, i.e. the distance between the openings in the template, is matched to the penetration depth of the fluorescence stimulation light. This means that the openings in the template are preferably at most so far away from each other that each volumetric element of the organ to be examined can be reached and recorded in terms of the detection of fluorescence signals. An operator thus knows that he/she can fully scan and pre-examine the entire organ in fluorescence terms if he/she simply uses all openings. However, this also means that the openings in the template are not applied arbitrarily close to each other in order to reduce both patient strain and the time required for the examination by reducing the number of puncture channels.

The terms “distally” and “proximally” should here denote a relative position that is distal or proximal of an operator of the system as a reference position. The terms “distal side” and “proximal side” should accordingly mean positions on a distal or proximal side of an object. The terms “distally” and “proximally” should here denote directions that extend distally and proximally.

The disclosure is explained below in more detail by way of examples of embodiment shown in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a, FIG. 1b and FIG. 1c are three schematic longitudinal section views of a first example form of embodiment of a biopsy system disclosed herein, each in different phases of use;

FIG. 2a, FIG. 2b and FIG. 2c are three further schematic longitudinal section views of the first example form of embodiment of a biopsy system disclosed herein in further phases of use;

FIG. 3a, FIG. 3b and FIG. 3c are three schematic longitudinal section views of a second example form of embodiment of a biopsy system disclosed herein, each in different phases of use;

FIG. 4a, FIG. 4b and FIG. 4c are three further schematic longitudinal section views of the second example form of embodiment of a biopsy system disclosed herein in further phases of use;

FIG. 5a, FIG. 5b and FIG. 5c are three schematic cross-sectional views of the first, second and third examples of embodiment of a biopsy system disclosed herein, each in different phases of use;

FIG. 6a, FIG. 6b and FIG. 6c, FIG. 7a, and FIG. 7b, FIG. 8a, FIG. 8b and FIG. 8c and FIG. 9 are schematic longitudinal section views of a third example form of embodiment of a biopsy system disclosed herein, each in different phases of use;

FIG. 10 is a schematic longitudinal view of a presented problem of the first, second and third example forms of embodiment of the biopsy system disclosed herein;

FIG. 11 is a schematic view of a fourth example form of embodiment of a biopsy system disclosed herein to solve the presented problem shown in FIG. 10; and

FIG. 12a, FIG. 12b and FIG. 12c, FIG. 13a, FIG. 13b and FIG. 13c, FIG. 14a, and FIG. 14b and FIG. 15a, and FIG. 15b are schematic longitudinal section views of a fourth example form of embodiment of a biopsy system disclosed herein, each in different phases of use; and

FIG. 16 is a schematic view of a fluorescence endoscopy element in accordance with an example of a biopsy system disclosed herein.

### DESCRIPTION OF PREFERRED EMBODIMENTS

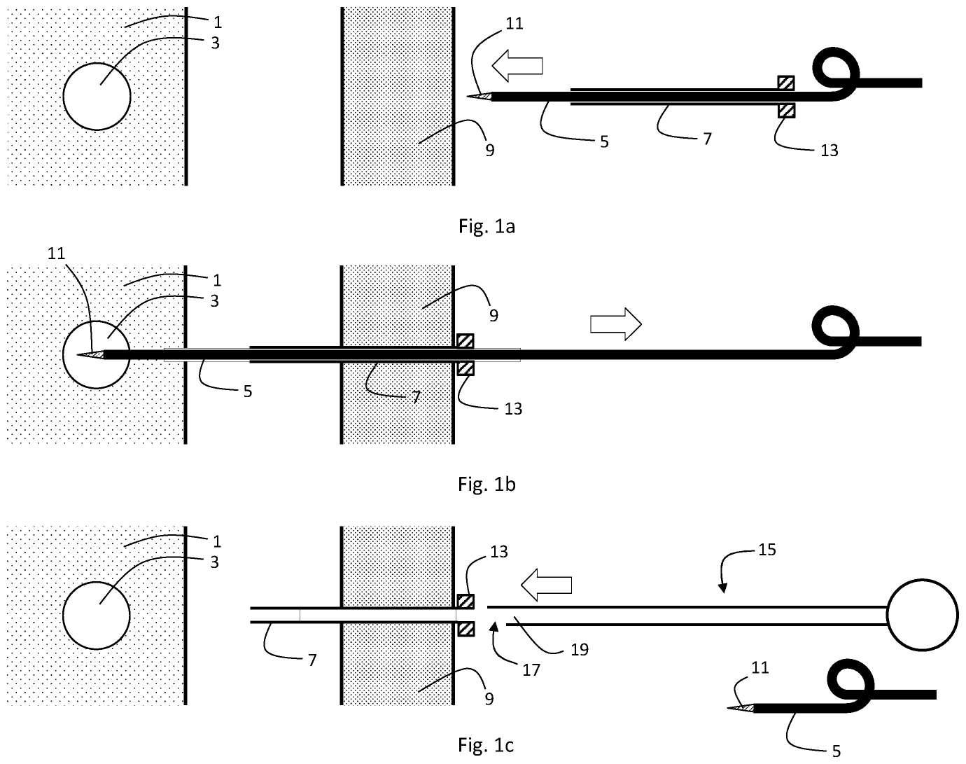

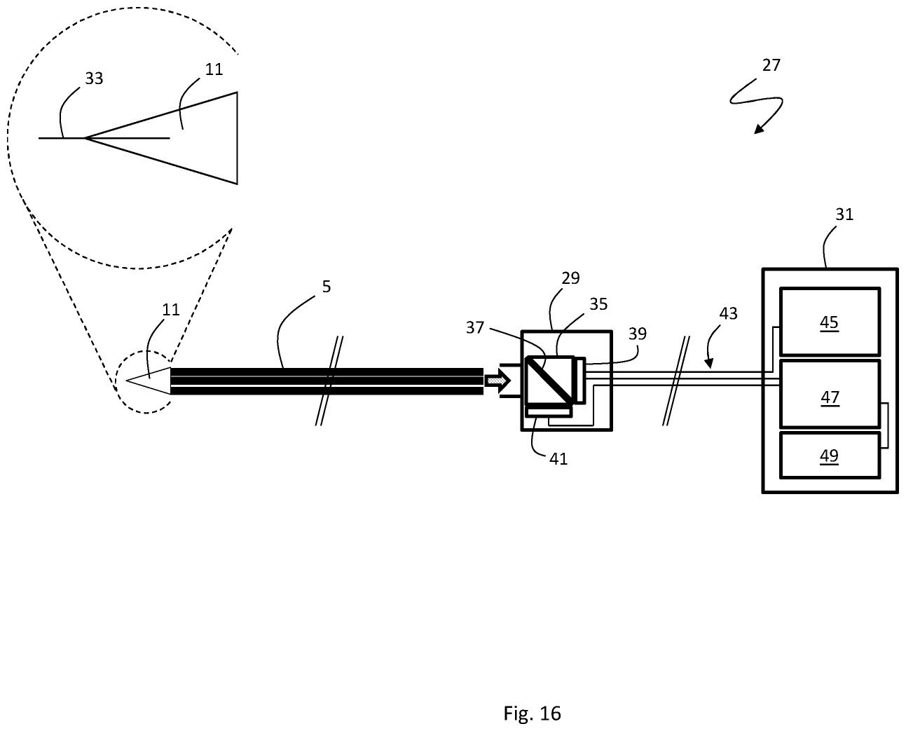

Referring to the drawings, FIGS. 1a-c show a biopsy system in the first three stages of use. In an organ 1 in the body of a patient, is tissue 3 to be examined, which appears abnormal, for example as a shadow, in an X-ray, CT and/or US image. For a more precise diagnosis, a tissue sample should now be taken from the tissue 3 to be examined, in order to be able pathologically examine it ex situ. The biopsy system used for taking the tissue sample (biopsy), has a distal section 16 of a fluorescence endoscopy element 27, shown in FIG. 16, which by means of an insertion sleeve 7 can be introduced percutaneously through the skin 9 of the patient in the direction of the tissue 3 to be examined. As shown in FIG. 1a shortly before insertion, the distal section 5 can be guided through the insertion sleeve 7 and is axially positionable relative to the insertion sleeve 7. The axial position of the distal section 5 can be adjusted as desired and fixed, for example through manual lateral pressing or other type of clamping (not shown). The distal section 5 has a distal tip 11, which on insertion projects beyond the distal end of the insertion sleeve 7, in order to make the insertion as atraumatic as possible. The insertion sleeve 7 can stabilize the distal section 5 against bending, particularly on the way through healthy, firm skin and/or muscle tissue 9. The handling of the distal section 5 is also facilitate by the insertion sleeve 7 located around the distal section 5. Insertion takes place preferably guided by a CT or US image, so that it can be observed whether the insertion is correctly positioned and aligned.

In FIG. 1b, the distal section 5 and the insertion sleeve 7 are already finally positioned. A proximal stop 13 on the insertion sleeve 7 is in contact with the skin 9 on the outside, so that the insertion sleeve 7 has a position of maximum penetration depth. The length of the insertion sleeve 7 is selected so that it does not reach the organ itself, but ends proximally thereof. The insertion sleeve 7 thus mainly supports the distal section 5 on the way through firm skin tissue 9. The distal section 5, on the other hand, is pushed distally further into the insertion sleeve 7, so tip 11 penetrates into the organ 1 and the tissue 3 to be examined. As shown in more detail in FIG. 16, the distal section 5 is part of the fluorescence endoscopy element 27 of the biopsy system and can comprise a light guide or light guide bundle for decoupling stimulation light and coupling in fluorescence light and to optically transmit the signal. The distal section 5 comprises proximal coupling in of stimulation light into the light guide, and at the distal tip 11, distal decoupling of the stimulation light from the light guide. At the distal tip 11, the distal section 5 also comprises distal coupling in of fluorescence light into the light guide, and proximal decoupling of the stimulation light from the light guide. This means that during insertion sleeve and axial positioning of the distal section 5 relative to the insertion sleeve 7, stimulation light can be distally decoupled at the distal tip 11 and at the same time fluorescence light can be proximally coupled in via the distal tip 11. The stimulation light can be shorter wave than the fluorescence light. Alternatively or additionally to a light guide or a light guide bundle, the distal section 5 of the fluorescence endoscopy element 27 can on the distal side comprise an LED for stimulation light decoupling and also on the distal site an image sensor or photodiode for coupling in fluorescence light and electronically transmitting the fluorescence signal.

So that malignant tissue 3 is clearly distinguished from healthy tissue by its fluorescence, the patient can be administered a photosensitizer or marker substance before the biopsy. The administration of a photosensitizer or marker substance may be dispensed with. Detected then is the fluorescence of the body's own pigments which, depending on the condition of the tissue, are present in different concentrations (autofluorescence). The amount of detected fluorescence is also a measure of the condition of the tissue here. The conspicuousness of the fluorescence signal can be seen, for example, in an increased or decreased fluorescence signal compared with healthy tissue. Thus, by way of the amplitude of the trapped fluorescence it can be seen in situ whether the distal tip 11 is located in malignant tissue 3. It can also be the case that the tissue 3 noticeable as shadowing in the CT or US image actually exhibits no conspicuousness in the fluorescence. In this case there is no need to take a biopsy from the tissue 3.

However, if a conspicuous fluorescence signal is recognized, through an axial movement of the distal tip 11, the size or the axial center of the fluorescing tissue 3 can be determined in that the distal tip 11 is positioned in such a way that the received fluorescence signal is maximally conspicuous. An operator can then read off or mark the axial position of the distal section 5 relative to the insertion sleeve 7 on a length marking on the distal section 5, for example. The distal section 5 can then be pulled out of the insertion sleeve 7 distally, wherein the insertion sleeve 7 remains in place.

In FIG. 1c, for the actual biopsy a biopsy element 15, for example in the form of a biopsy hollow needle, is placed on the insertion sleeve 7. Here, the biopsy element 15 has a bevelled distal end 17 which forms a distal hollow chamber 19 for receiving tissue samples. If the distal end 17 is inserted into tissue and/or peels this off through a back and forth movement, a tissue sample remains in the hollow chamber 10 and can be removed with the biopsy element 15. For example, via a distally beveled cylinder which fills the hollow chamber and can be moved back and forth from the proximal side by the operator and in this way blocks and/or releases the hollow chamber in a controlled manner, it can be determined by the operator from which axial point in the tissue a tissue sample is taken. The above-described biopsy element 15 and its function act as an example of a possible tissue-taking procedure. Other forms of taking tissue, e.g., via laterally applied notches on the distal end of the biopsy element 15 are also possible.

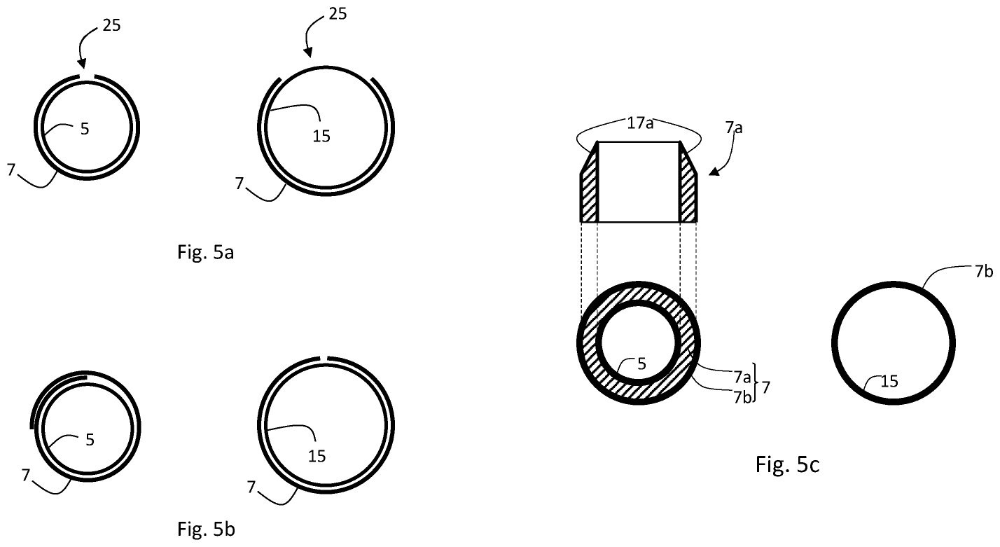

The insertion sleeve 7 now has the effect that the biopsy element 15 can be precisely positioned at the point at which the fluorescence endoscopy element has detected a conspicuous fluorescence signal. It also helps the biopsy element 15 when it is inserted through the skin tissue 9. Through this, the insertion of the biopsy element 15 is considerably less traumatic. As shown in FIGS. 5a-c, the biopsy element 15 can certainly be much thicker than the distal section 5 in order to be able to take a sufficiently large tissue sample for the pathological examination. In this case, the insertion sleeve 7 can preferably expand radially in order to take up the biopsy element 15. Radial expansion is less traumatic than directly inserting the biopsy element 15. The read-off or marked axial position of the distal section 5 relative to the insertion sleeve 7 can now be reproduced by means of corresponding length markings on the biopsy element 15.

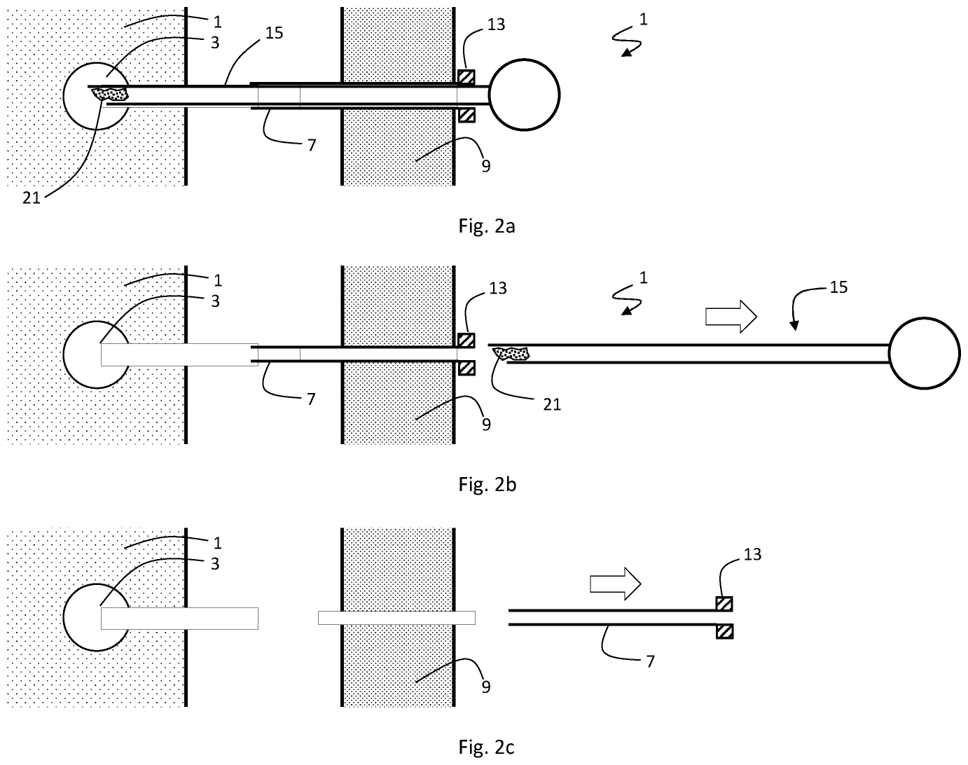

In FIG. 2a, the reproduced axial position of the biopsy element 15 is shown. The distal end 17 of the biopsy element 15 projects by the same length beyond the distal end of the insertion sleeve into the tissue 3 to be examined in the organ 1. Through this, in the distal hollow chamber 19, as soon as this is released by a corresponding axial movement of the solid cylinder (not shown) in the hollow chamber 19 in the proximal direction by the operator, a tissue sample 21 of the tissue 3 to be examined is caught. FIG. 2b shows how the biopsy element 15 with the tissue sample 21 is proximally pulled out of the insertion sleeve 7 again. The taken tissue sample 21 can now be examined pathologically ex situ. Finally, as shown in FIG. 2c, the insertion sleeve 7 is proximally pulled back out of the body of the patient.

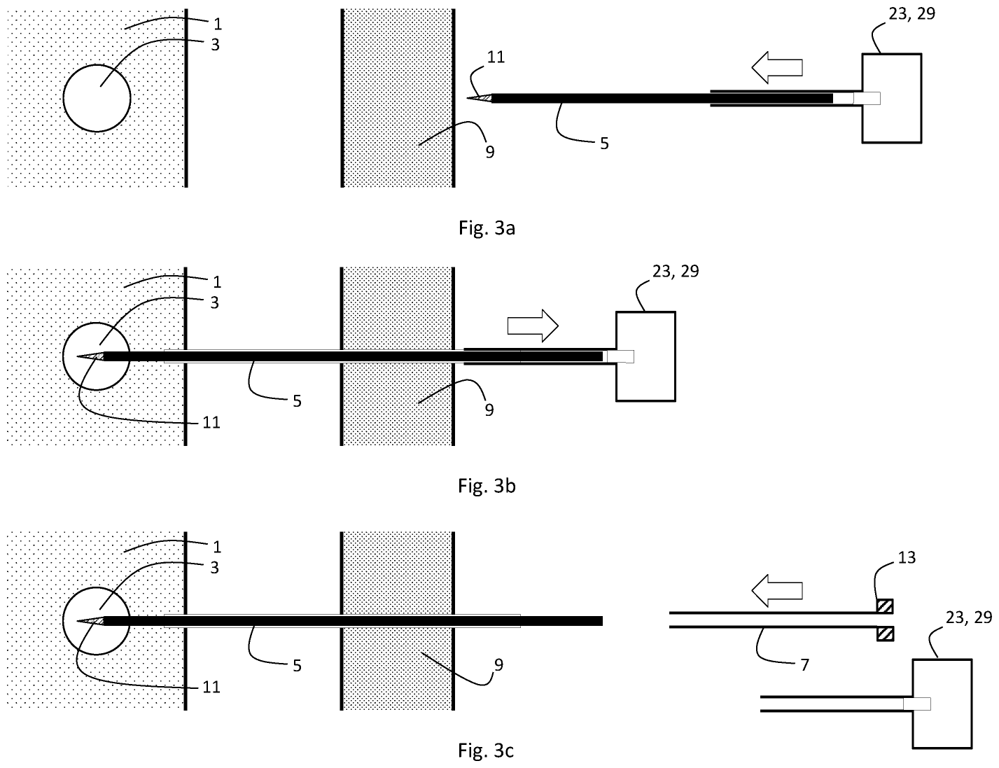

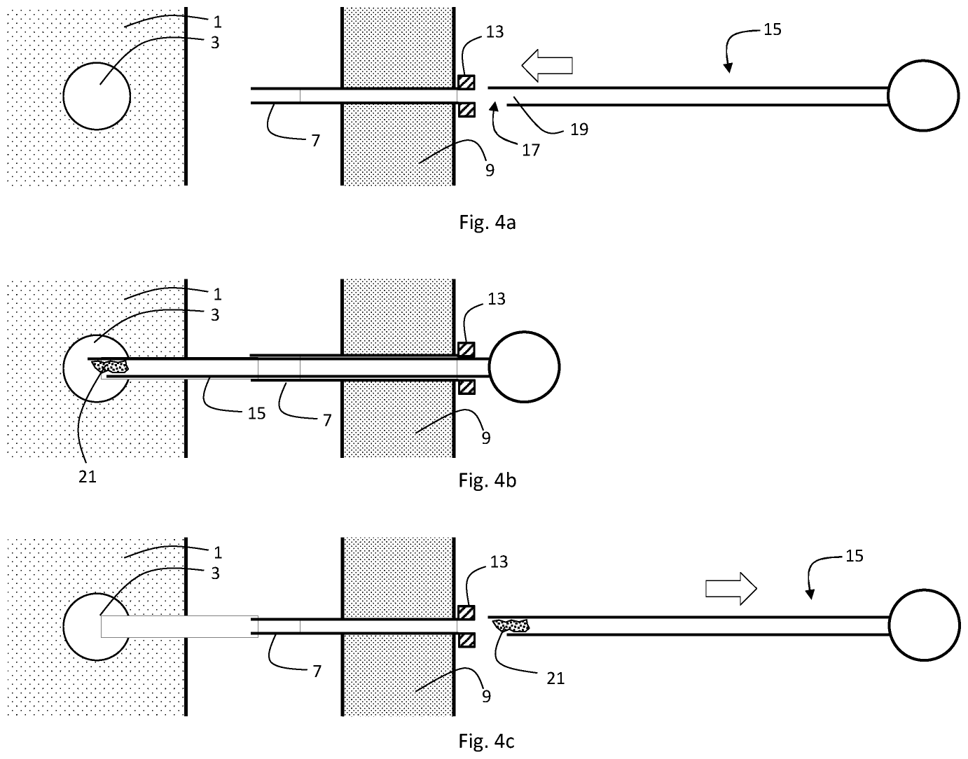

In contrast to the above-described example of embodiment, in the example of embodiment shown in FIGS. 3a-c and FIGS. 4 a-c, the distal section 5 more rigid, for example reinforced by a thin-walled tube. Through this, the distal section 5 does not need the insertion sleeve 7 as an aid for insertion through the firm skin tissue 9, but is itself rigid enough for this. A connectable and disconnectable handling device 23 can, as here, be provided to allow the manual handling, positioning and alignment of the distal section 5. Preferably an optical component 29 is integrated into the handling device 23 for coupling in fluorescence light as part of the fluorescence endoscopy element.

The insertion sleeve 7 is only used when by means of the fluorescence signal a final position of the distal section 5 with its distal tip 11 in the middle of the tissue 3 to be examined has been found and set. The handling device 23 or the optical component 29 can then be disconnected (see FIG. 3b), so that from proximal the insertion sleeve 7 can be pushed over the distal section 5 (see FIG. 3c). As soon as the insertion sleeve 7 is in contact on the skin 3 with the stop 13 and thus finally positioned, the distal section 5 can be pulled out proximally, and, as shown in FIGS. 4a-c, the actual biopsy can be performed with the biopsy element 15 as described above. The most recently described procedure, i.e., the procedure as shown in FIGS. 3a-c and 4a-c, has the advantage over the procedure described in FIGS. 1a-c and 2a-c, that firstly conspicuous tissue can be found faster and secondly with less burden on the patient (i.e. less traumatically), as for this procedure the insertion sleeve 7 is not yet used. This advantage becomes particularly relevant, if for the detection of conspicuous fluorescence, the distal section 5 has to be inserted into the skin 9 at several different places.

FIGS. 5a-c show three forms of embodiment the insertion sleeve 7. In FIGS. 5a-c, on the left in each case, the insertion sleeve 7 is shown with the inserted distal section 5, and on the right the insertion sleeve 7 with the inserted biopsy element 15. In each case the biopsy element 15 is thicker than the distal section 5, i.e. it therefore has a greater radial extent. In the forms of embodiment according to FIG. 5a,b, the insertion sleeve 7 is radially expandable and slit. In the first form of embodiment according to FIG. 5a, a slit 25, which essentially extends over the entire length of the insertion sleeve 7, expands when the thicker biopsy element 15 is inserted, and elastically contracts when the thinner distal section 15 or nothing is inserted.

In the second form of embodiment shown in FIG. 5b, the insertion sleeve 7 is configured to overlap in the circumferential direction when the thinner distal section 5 or nothing is introduced. With the inserted biopsy element 15, the overlapping is smaller, or, as shown in FIG. 5b on the right, no longer present. If the difference of the radial expansions between the distal section 5 and the biopsy element 15 is large enough, it can also be the case that the insertion sleeve 7 with inserted distal section 5 is overlapping as shown in FIG. 5b on the left, and with the inserted biopsy element 15 expanded with expanded slit 25 as shown in FIG. 5a on the right.

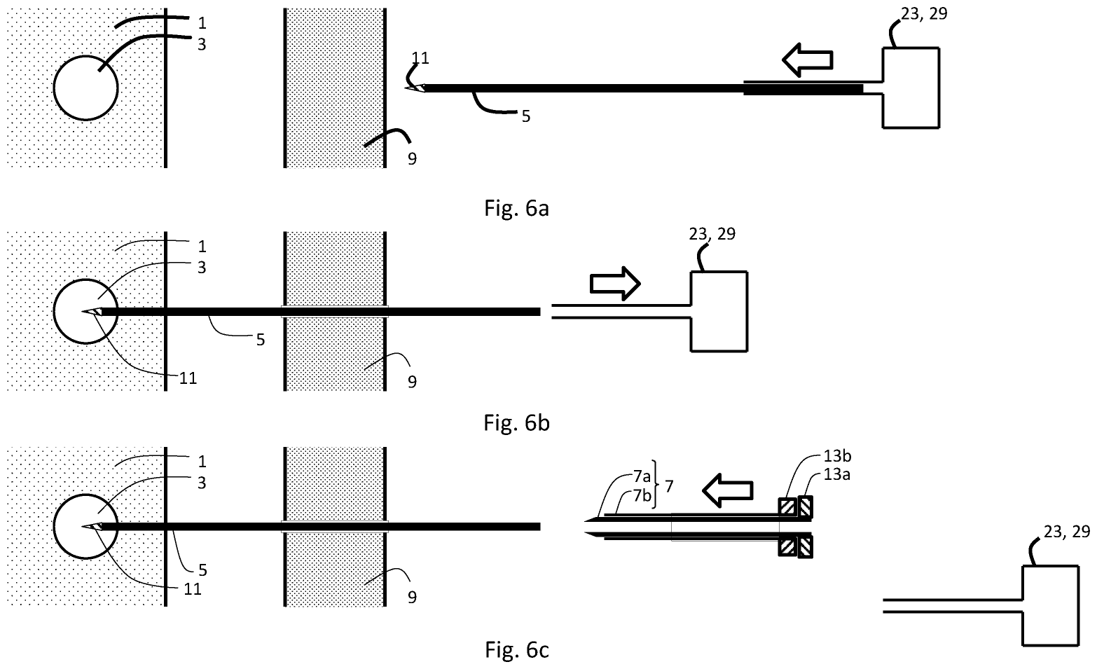

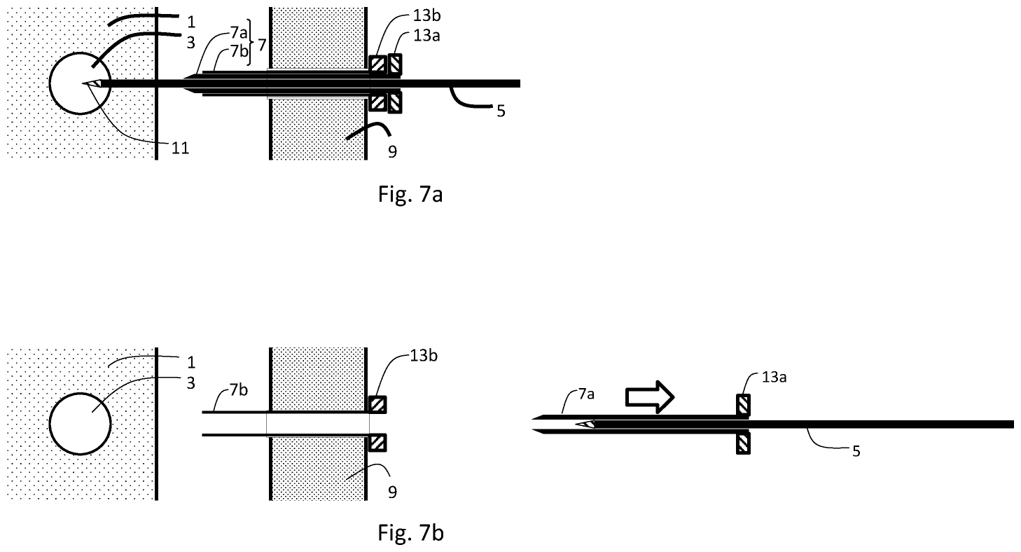

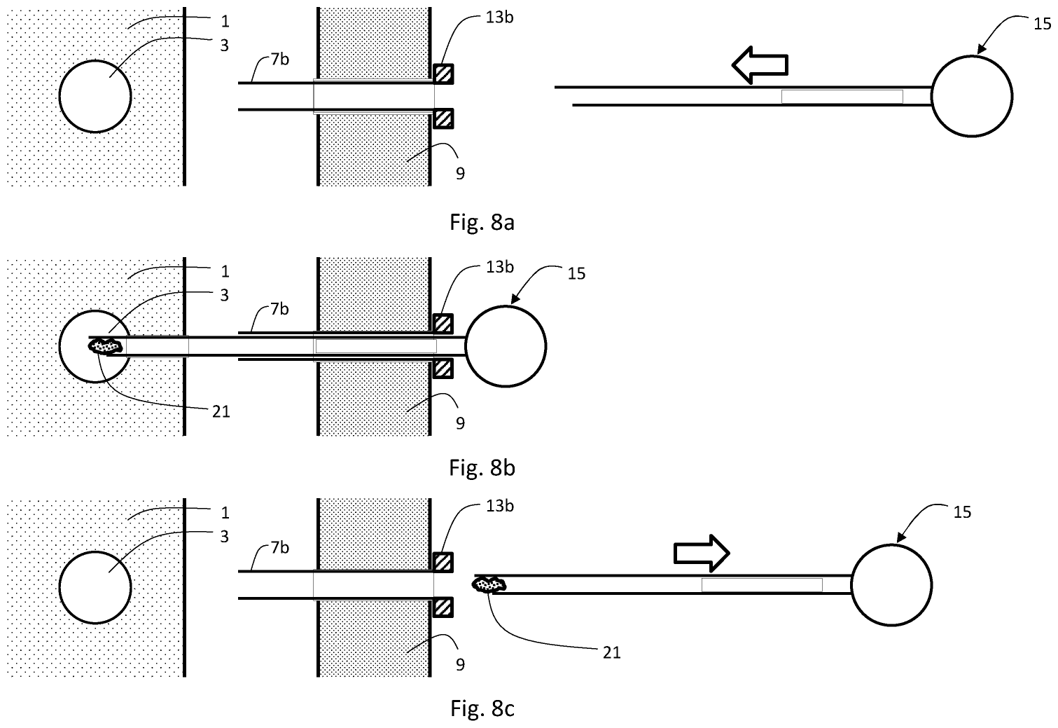

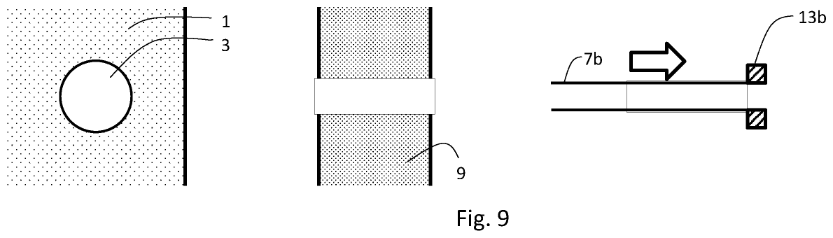

In the third form of embodiment shown in the FIG. 5c, the insertion sleeve 7 has at least two separate parts, namely an inner sleeve 7a and an outer sleeve 7b which are insertable into each other, detachable from one another and displaceable with regard to each other (see also FIGS. 6a-c, 7a,b, 8a-c und 9). As long as the thinner distal section 5 is still inserted in the body, the inner sleeve 7a is also inserted into the outer sleeve 7b, in order in this way to guide the distal section 5, i.e. to support it in the radial direction. In order to insert the thicker biopsy element 15 and to create the space necessary for this in the radial direction, the inner sleeve 7a is pulled out of the outer sleeve 7b in the proximal direction, wherein the outer sleeve 7b remains in place. The subsequently inserted, thicker biopsy element 15 is then guided by the outer sleeve 7b, i.e. it is supported by this in the radial direction. This wall thickness of the inner sleeve 7a is such that it essentially corresponds to the difference between the outer diameter of the biopsy element 15 and the outer diameter of the distal section 5.

Preferably the inner sleeve 7a distally has a cone-shaped endpiece 17a in order to make largely atraumatic introduction into the tissue possible. The left top illustration of FIG. 5c shows a longitudinal section through the distal end of the inner sleeve 7a.

Compared to the two previous forms of embodiment, the third form of embodiment has the advantage that almost any size difference between the distal section 5 and the biopsy element 15 in terms of their radial extent ca be handled, in that the wall thickness of the inner sleeve 7a is configured to be accordingly thick, i.e. adapted to the diameter difference and with an appropriately pointed and in this sense atraumatic endpiece 17a. In contrast, the expansion of the insertion sleeve 7 in the previous forms of embodiment is restricted due to the limited elasticity of the insertion sleeve 7. Through this, the advantage of the biopsy system described here to find and biopsy many lesions with as little tissue damage as possible is implemented in the best possible way. With the very thin distal section 5, conspicuous fluorescence can be sought extensively and closely, yet in manner that is gentle on the patient, as a very thin distal section only causes little damage in healthy tissue. Subsequently, with a comparatively thick biopsy element 15, which only has to be brought to the site of the abnormal fluorescence, a relatively large, and therefore valuable for the pathologist, tissue sample can be obtained.

Compared to the two previous forms of embodiment, the third form of embodiment has the further advantage that on insertion of the biopsy element 15 no force for widening the insertion sleeve 7 has to be applied and also on pushing forward the biopsy element no frictional force on the insertion sleeve 7 compared to the previous forms of embodiment has to be overcome.

As in the second example of embodiment according to FIGS. 3a-c and 4a-c, in the third example of embodiment shown in FIGS. 6a-c, 7ab, 8a-c and 9, the distal section 5 is reinforced by a thin-walled tube, so that no insertion aid through the firm skin tissue 9 is required. Also in this third example of embodiment, the optical component 29 is preferably configured in such a way that it takes over the tasks of the handling device 23 in FIGS. 3a-c and a separate, additional handling device 23 is no longer needed. The insertion sleeve 7, here in the form of a double sleeve consisting of an outer sleeve 7a and inner sleeve is also only used when the final position of the distal section 5 is reached, see FIG. 6c.

Initially, both sleeves 7a and 7b are brought into position, i.e., introduced so far into the tissue until their stops 13a and 13b are in contact on the skin tissue, see FIG. 7a. The distal section 5, together with the inner sleeve 7a is then pulled out of the human body, i.e., out of the outer sleeve 7b, see FIG. 7b, in order to create the access for the thicker biopsy element 15.

This thicker biopsy element 15 can then be introduced into the thus provided lumen (see FIG. 8a), in order to finally take a tissue sample 21 (see FIG. 8b). The biopsy element 15 together with the tissue sample 21 is then taken out of the human body/the outers sleeve 7b in order to forward it to the pathologist. Finally the outer sleeve 7b is removed (see FIG. 9).

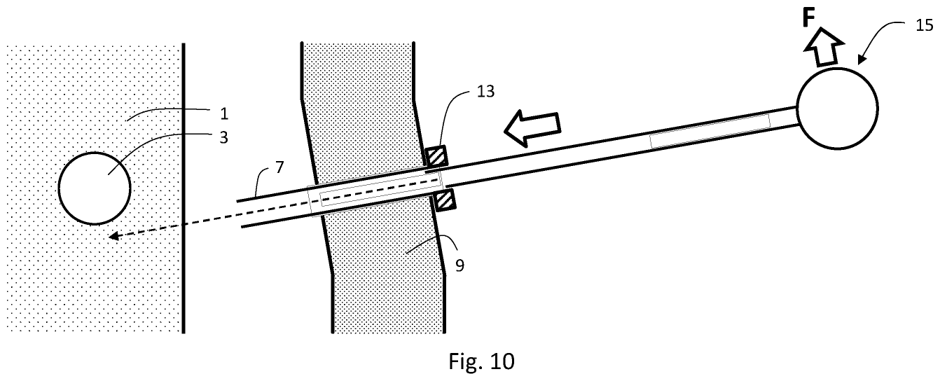

Shown in FIG. 10, is a problem that can occur in the previously described three forms of embodiment of the biopsy system. The skin 9 may, at a point of the body of the patient, be so flexible that laterally acting, non-axial forces F exert lever forces on the insertion sleeve 7, which can change the orientation and/or position of the insertion sleeve 7 relative to the tissue 3 to be examined. However, if the insertion sleeve 7 does not retain the correct orientation and/or position relative to the tissue 3 to be examined, there is a risk of the biopsy element 15 missing the tissue 3 to be examined. This is shown in FIG. 10.

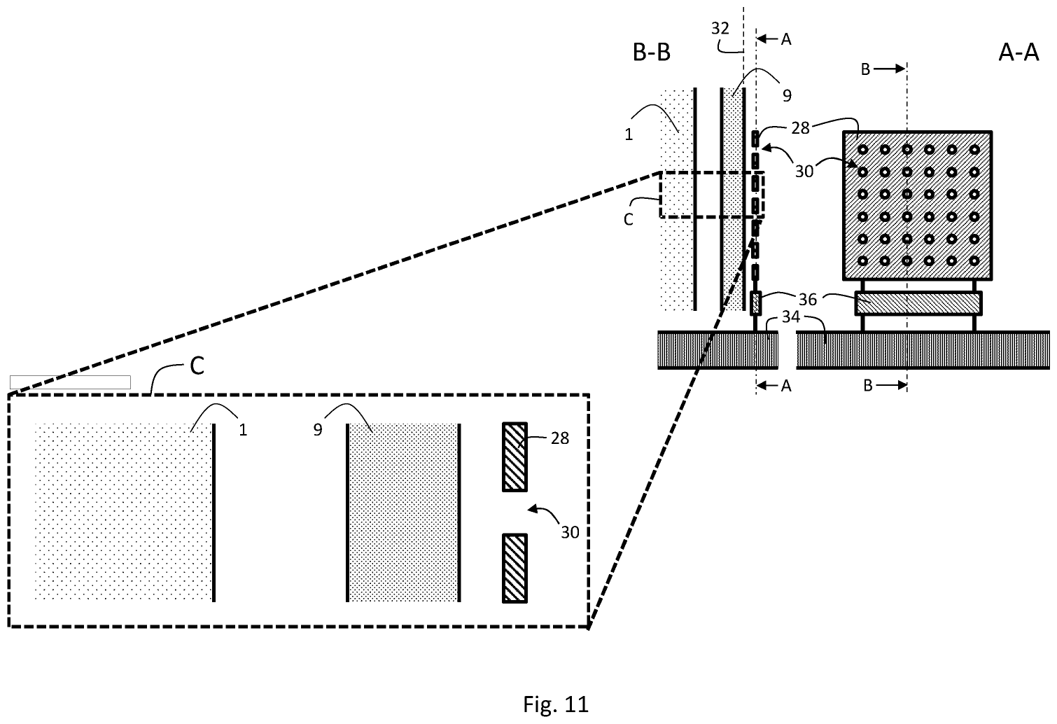

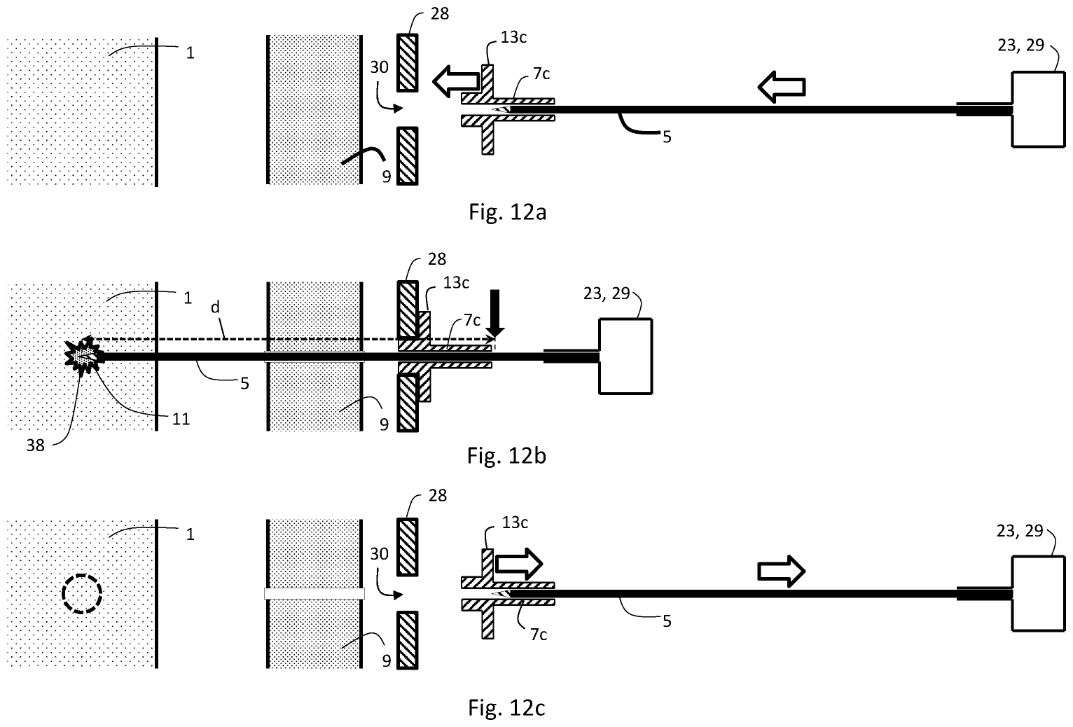

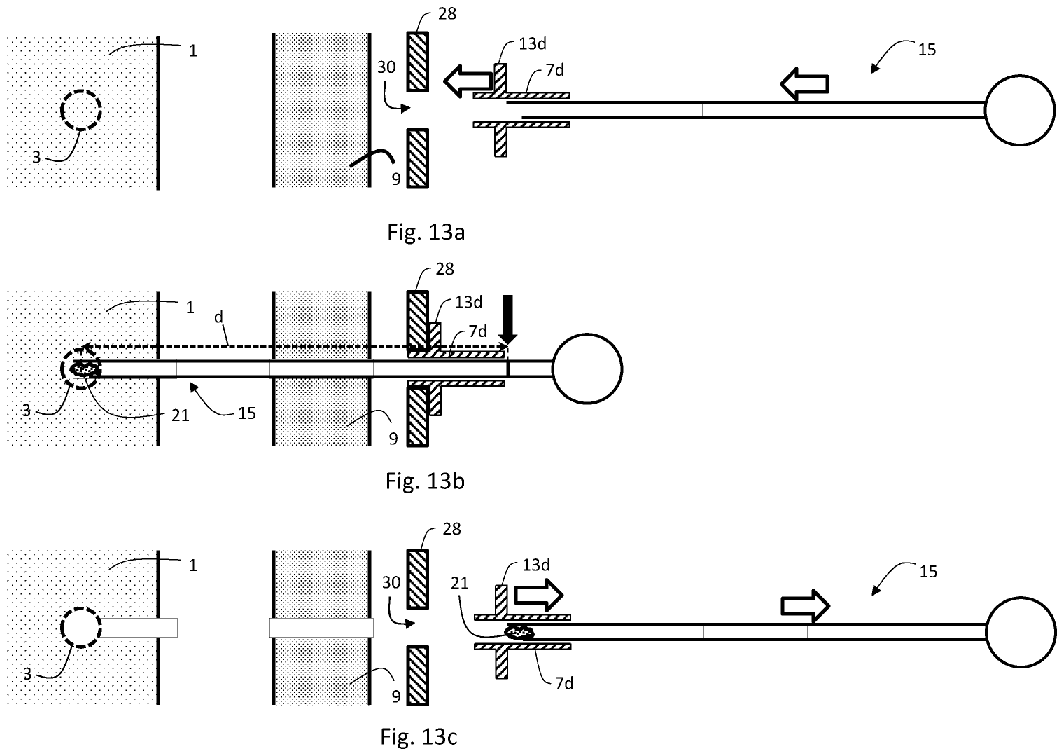

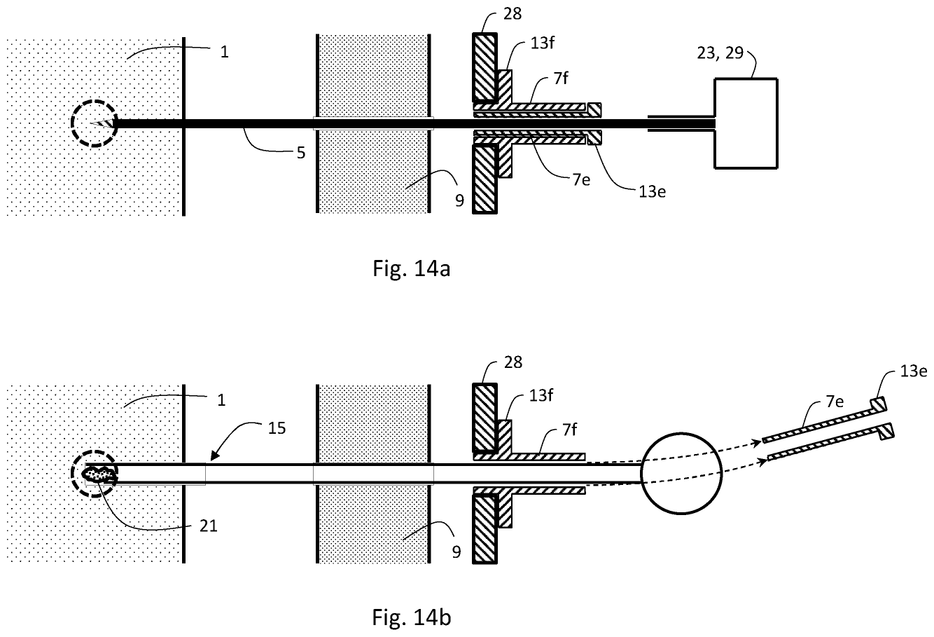

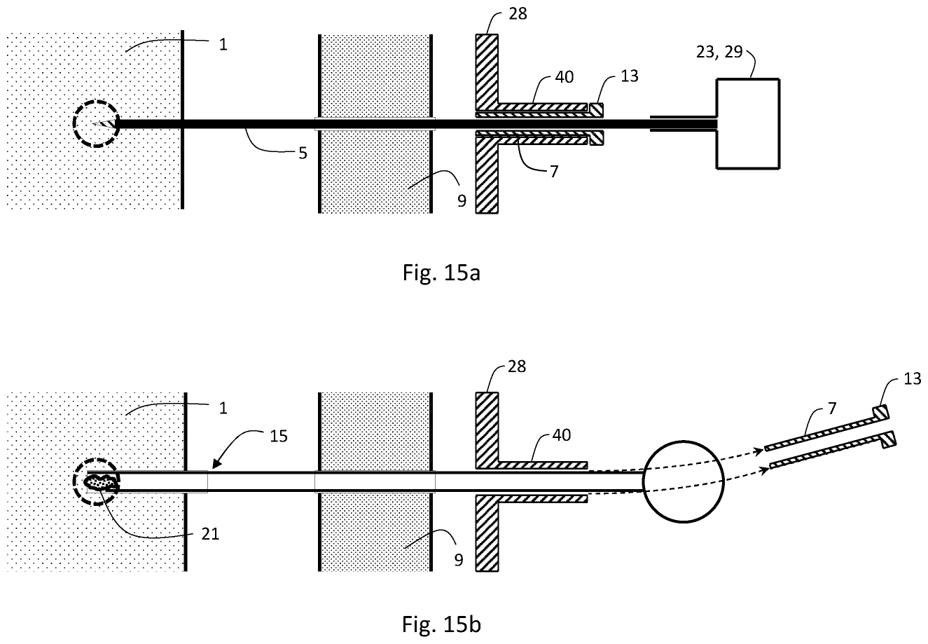

FIGS. 11, 12a-c, 13a-c, 14a,b and 15a,b show various versions of a fourth form of embodiment of the biopsy system which also comprises a template 28. The template 28 is shown here as a perforated plate with a plurality of openings 30 distributed in a grid-like manner. The template 28 is preferably rigid, at least much firmer than the skin 9 and is fixed relative to the body 32 of the patient. For this it can be stuck to the skin 9 and/or, as shown in FIG. 11, fixed on an operating table 34, on which the body 32 of the patient has a defined position. The template 28 is preferably flat in direct contact with skin 9 of the body 32 of the patient. By way of a positioning unit 36, the template 28 is here positionable preferably in three different spatial directions in a translational and/or rotational manner, i.e., with six degrees of freedom, and fixable in a selected position and orientation relative to the operating table 34.

FIGS. 12a-c show how conspicuously fluorescing tissue 3 in the organ 1 is found with the fluorescence endoscopy element 27, which comprises the distal section 5 and an optical component 29 including handling device 23. The operator is not now fully free to decide about the insertion position and angle, but chooses an opening 20 in the template 28 for insertion for the distal section 5. Through the selected opening 30, the insertion position and the insertion angle are fully defined and with the penetration depth the operator still only as one degree of freedom. As the openings 30 in the template 28 have to be large enough so that the thicker biopsy element 15 fits through later, the insertion sleeve 7 here acts as an adapter between the distal section 5 and/or biopsy element 15 and the template 28. The insertion sleeve 7 is not here inserted into the skin 9 of the patient which is additionally gentle on the patient.

In this variant the insertion sleeve 7 has two parts, wherein a first part 7c acts as an adapter between the distal section 5 and the template 28 and a second part 7d (see FIGS. 13 a-c) acts as an adapter between the biopsy element 15 and the template 28. The first insertion sleeve part 7c fixes the distal section 5 laterally in the selected opening 30 in the template 28 and is in contact on the template 28 with a flange acting as stop 13c. Locking means, such as a bayonet lock or Luer connection can also be provided for a temporary positive and/or non-positive connection between the first insertion sleeve part 7c and the template 28.

As shown in FIG. 12b, the distal section 5 is inserted through the insertion sleeve part 7c as well as through the skin 9 into the organ 1 to be examined in order to search there for conspicuous fluorescence 38 at different penetration depths. If, at penetration depth d (measured at the proximal end of the first insertion sleeve part 7c), conspicuous fluorescence 38 is found, the penetration depth d can be read off and noted using the length markings on the distal section 5. Alternatively or additionally, a displaceable marking element can be pushed to the proximal end of the first insertion sleeve part 7c and axially fixed on the distal section 5. Then, as shown in FIG. 12c, the distal section 5 and the first insertion sleeve part 7c is pulled proximally out of the template 28.

The actual biopsy is shown in FIGS. 13a-c. Into the same opening in which the distal section 5 previously was, the biopsy element 15 is introduced, wherein a second insertion sleeve part 12d acts as an adapter between the biopsy element 15 and template 28. The second insertion sleeve part 7d has the same outer diameter matched to the opening as the first insertion sleeve part 7c. However, as the biopsy element 15 is thicker than the distal section 5, the inner diameter of the second insertion sleeve part 7d is correspondingly larger than the inner diameter of the first insertion sleeve part 7c. The axial length of the first insertion sleeve part 7c and the second insertion sleeve part 7d is identical. The biopsy element 15 can now be inserted by the noted penetration depth d into the organ 1 in order to take a tissue sample 21 from the tissue 3 to be examined in the area of conspicuous fluorescence 38.

A second variant of the fourth form of embodiment of the biopsy system is shown in FIG. 14a,b, wherein FIG. 14a shows the localization by way of the fluorescence endoscopy element 27 and FIG. 14b the biopsy by means of biopsy element 15. In contrast to the above-described variant according to FIGS. 12a-c and 13a-c, here the two part of the insertion sleeve 7 are configured as inner sleeve 7e and outer sleeve 7f. The outer diameter of the inner sleeve 7e is matched to the inner diameter of the outer sleeve 7f as well as the outer diameter of the biopsy element 15. The inner diameter of the inner sleeve 7e is matched to the outer diameter of the distal section 5. The outer diameter of outer sleeve 7f is matched to the inner diameter of openings 30 in the template 28. Inserted into each other, the inner sleeve 7e and the outer sleeve 7f function as a suitable adapter between distal section 5 and template 28. The outer sleeve 7f along with proximally pulled out inner sleeve 7f functions as a suitable adapter between biopsy element 15 and template 28.

A third variant of the fourth form of embodiment of the biopsy system is shown in FIG. 15a,b, wherein FIG. 15a shows the localization by way of the fluorescence endoscopy element 27 and FIG. 15b the biopsy by means of biopsy element 15. Here, the insertion sleeve 7 is in one piece or only consists of an inner sleeve which acts as an adapter between the distal section 5 and the template 28. A separate outer sleeve for guiding the biopsy element 15 is not required here as the inner diameter of the openings 30 is already matched to the outer diameter of the biopsy element 15 and the template 28 in proximal extension around the openings 30 has a guide element 40 for the biopsy element 15. Alternatively, at a certain axial thickness of the template 28, the guide element 40 can be formed by the opening 30 itself.

If, with regard to the penetration depth of the fluorescence stimulation light, the grid size is sufficiently small, i.e. if the openings 30 are close enough to each other so that with the distal section 5 sequentially introduced into the openings 30 each volumetric element of the organ to be examined is reached with fluorescence stimulation light and also in an examination each opening 30 is actually used over the entire organ depth, it is also ensured that each volumetric element of the organ to be examined is pre-examined in terms of fluorescence, i.e. no volumetric element is disregarded or forgotten.

FIG. 13 schematically shows the fluorescence endoscopy element 27 in more detail. The fluorescence endoscopy element 27 comprises the distal section 5, an optical component 29 and an operating unit 31, wherein the optical component 29 preferably takes over the function of a previously described handling device 23. On the proximal side, the distal section 5 is connectable or firmly connected to the optical component 29, through which proximal decoupling of fluorescence light from the light guide of the distal section 5 into the optical component 29, as well as distal coupling in of stimulation light from the optical component 29 into the light guide is achieved. The distal tip 11 of the distal section 5 can be configured as a transparent needle tip that decouples stimulation light from the light guide and couples fluorescence light into the light guide. To reinforce the tip 11 and to reduce tissue trauma through insertion of the distal section 5, the tip 11 can, as shown, be provided with a spike 33 which can project beyond the distal needle tip.

The optical component 29 comprises a beam splitter 35 in the form of a beam splitter block formed by a pair of prisms. The prisms each adjoin each other with optical interfaces, that extend at an angle of 45° in relation to the optical axis. Arranged between or at least at or on one of the optical interfaces is a longpass filter 37 which essentially transmits longer wave fluorescence light and reflects shorter wave stimulation light. Essentially coaxially to the optical axis, the beam splitter 35 thus has an image path side, on which an image sensor 39, a photodiode or similar is arranged. Laterally, the beam splitter 35 has an illumination path side on which a light source 41, for example in the form of an LED or laser decoupling, is arranged. The longpass filter 37 reflects the stimulation light distally to the distal section 5. Preferably the optical component 29 is integrated into a handle or a similar ergonomic component which can be securely, but detachably, locked on the distal section 5 so that it can also take on the tasks/functions of the handling device 23. In this way it is firstly possible to monitor in fluorescence optical terms the entire movement procedure of the distal section 5 in the tissue in relation to searching for pathologically relevant material, and secondly the handling device 23a as such or replacement thereof with the optical component 29 can be dispensed with.

The fluorescence endoscopy element 27 is preferably set up to optionally couple in a first stimulation light and/or a second stimulation light proximally, wherein the first stimulation light is on average shorter wave than the second stimulation light. The fluorescence endoscopy element 27 is also configured to optionally produce a first image or first fluorescence signal from a first tissue area with the first fluorescence light stimulated with the first stimulation light or to receive a second image of a second fluorescence signal from a second tissue area from second fluorescence light stimulated with the second stimulation light, wherein the first image or the first fluorescence signal originates from a smaller tissue volume than the second image or the second fluorescence signal, but thereby has a higher spatial resolution.

The operating unit 31 is signal-connected or connectable to the image sensor 39, the photodiode or similar, and the light source 41 via a cable connection 43. The light source 41 can be controlled via the operating unit 31 and the signals of the image sensor 39, the photodiode or similar, can be received, processed and, if necessary, visualized by the operating unit 31. For this, the operating unit has an optical and/or acoustic display component 45, a control and supply component 47 and an operating component 49.

The numbering of the components or movement directions as “first”, “second”, “third” etc. is purely arbitrarily selected to distinguish the components or movement directions from each other and can be selected in any other way. No order of priority is associated therewith. The designation of a component or technical feature as “first” should not be misunderstood to the effect that there has to be a second component or technical feature of this type. Moreover, any process steps, unless explicitly mentioned otherwise or absolute necessary, can be carried out in any order and/or partially or fully overlapping in time.

Equivalent forms of embodiment of the parameters, components or functions described herein, which in consideration of this description appear evident to a person skilled in the art, are included herein as if they have been explicitly described. Accordingly, the protective scope of the claims should cover such equivalent forms of embodiment. “Can” features designated as optional, advantageous, preferred, desired or similar are to be understood as optional and not as restricting the protective scope.

The described forms of embodiment are to be understood as illustrative examples and do not represent an exhaustive list of possible forms of embodiment. Each feature disclosed as part of a form of embodiment can be used alone or in combination with one or more other features, irrespective of in which form of embodiment the features have been described in each case. Whereas at least one example of embodiment is shown and described here, derivations and alternative forms of embodiment that in consideration of this description appear evident to a person skilled in the art, are included in the protective scope of this disclosure. Furthermore, neither the term “comprise” should exclude additional, other features or process stages, nor should “a” or “an” exclude a plurality.

While specific embodiments of the invention have been shown and described in detail to illustrate the application of the principles of the invention, it will be understood that the invention may be embodied otherwise without departing from such principles.

### LIST OF REFERENCES

1 Organ
3 Tissue to be examined
5 Distal section of the fluorescence endoscopy element
7 Insertion sleeve
7a, f Inner sleeve
7b, f Outer sleeve
7c First insertion sleeve part
7d Second insertion sleeve part
9 Skin/skin tissue
11 Distal tip of the distal section of the fluorescence endoscopy element
13 Insertion sleeve stop
13a,e Inner sleeve stop
13b,f Outer sleeve stop
13c First insertion sleeve part stop
13d Second insertion sleeve part stop
15 Biopsy element
17 Distal end of the biopsy element
17a Cone-shaped end piece
19 Hollow chamber of the biopsy element
21 Tissue sample
23 Handling device
25 Slit
27 Fluorescence endoscopy element
28 Template
29 Optical component
30 Openings
31 Operating unit
32 Body of the patient
33 Spike
34 Operating table
35 Beam splitter
36 Positioning unit
37 Longpass filter
38 Conspicuous fluorescence
39 Image sensor, photodiode or similar
40 Guide element
41 Light source
43 Cable connection
45 Display component
47 Control and supply component
49 Operating component

1. A biopsy system comprising:
a fluorescence endoscopy element;
a biopsy element for taking a sample of the tissue to be examined from an organic body; and
an insertion sleeve for placing a distal end of the fluorescence endoscopy element and/or the biopsy element in or on the tissue to be examined in the organic body,
wherein the fluorescence endoscopy element comprises a distal section with distal decoupling of stimulation light as well as distal coupling in of fluorescence light,
wherein the distal section of the fluorescence endoscopy element is axially moveably positioned relative to the insertion sleeve and can be proximally removed from the insertion sleeve,
wherein the biopsy element, with the distal section of the fluorescence endoscopy element is removed, is configured to be placed through the insertion sleeve into or onto the tissue to be examined in the organic body.

2. The biopsy system according to claim 1, wherein the insertion sleeve is more rigid than the distal section of the fluorescence endoscopy element.

3. The biopsy system according to claim 1, wherein on the proximal side, the insertion sleeve can be everted over the distal section of the fluorescence endoscopy element.

4. The biopsy system according to claim 1, wherein the biopsy element has a greater radial extent than the distal section of the fluorescence endoscopy element and an inner diameter of the insertion sleeve is adjustable for optionally receiving the distal section of the fluorescence endoscopy element and the biopsy element.

5. The biopsy system according to claim 4, wherein the insertion sleeve is axially slit and expandable.

6. The biopsy system according to claim 5, for receiving the distal section of the fluorescence endoscopy element, the insertion sleeve is configured in a circumferentially overlapping manner, wherein when the biopsy element is received, the overlapping is smaller or no longer exists.

7. The biopsy system according to claim 4, wherein the insertion sleeve comprises an inner sleeve and an outer sleeve, wherein for receiving the biopsy element, the inner sleeve is removable in the proximal direction from the outer sleeve.

8. The biopsy system according to claim 7, wherein the inner sleeve has an inner diameter that essentially corresponds to an outer diameter of the distal section of the fluorescence endoscopy element, and the outer sleeve has an inner diameter that essentially corresponds to an outer diameter of the biopsy element.

9. The biopsy system according to claim 1, wherein the distal section of the fluorescence endoscopy element as well as the biopsy element have longitudinal markings and/or an axially positionable stop for a defined reproducible relative axial position of the distal section or the biopsy element relative to the insertion sleeve.

10. The biopsy system according to claim 1, wherein the fluorescence endoscopy element is configured to optionally proximally couple in a first stimulation light and/or a second stimulation light, wherein the first stimulation light is on average shorter wave than the second stimulation light, and wherein the fluorescence endoscopy element is configured to optionally produce a first fluorescence signal from a first tissue area with the first fluorescence light stimulated with the first stimulation light and to receive a second fluorescence signal from a second tissue area from second fluorescence light stimulated with the second stimulation light.

11. The biopsy system according to claim 1, wherein the distal section of the fluorescence endoscopy element comprises a light guide with proximal coupling in of stimulation light and/or proximal decoupling of fluorescence light, wherein the distal end of the fluorescence endoscopy element distally decouples the stimulation light from the light source and/or distally couples the fluorescence light into the light guide.

12. The biopsy system according to claim 11, wherein proximally, the fluorescence endoscopy element comprises an optical component which is detachably connectable or firmly connected to the distal section of the fluorescence endoscopy element, wherein the optical component comprises a beam splitter with an image path side and an illumination path side, wherein to decouple the fluorescence light an image sensor or a photodiode is detachably connectable or firmly connected to the image path side, and wherein to couple in the stimulation light, a light source is detachably connectable or firmly connected to the illumination path side.

13. The biopsy system according to claim 12 wherein the optical component has an optical longpass filter which transmits the fluorescence light more strongly to the image path side than the stimulation light, and/or comprises an optical shortpass filter which reflects the fluorescence light more strongly to the image path side than the stimulation light.

14. The biopsy system according to claim 12, wherein the fluorescence endoscopy element comprises an operating unit signal-connected or connectable with the image sensor or the photodiode and/or the light source, wherein the operating unit comprises an optical or acoustic display component, a control and supply component and an operating component.

15. The biopsy system according to claim 1, further comprising a template, positionable and fixable relative to the organic body, with at least one opening for selectively receiving the insertion sleeve and the biopsy element, wherein the insertion sleeve and/or the biopsy element in the at least one opening (30) is fixed against lateral displacement and tilting through the template.

16. The biopsy system according to claim 15, wherein the insertion sleeve comprises a first part and a second part, wherein the first part fixes the distal section of the fluorescence endoscopy element in the at least one open against lateral displacement and tipping, and wherein the second part fixes the biopsy element in the at least one opening against lateral displacement and tipping.

17. The biopsy system according to claim 15, wherein the template comprises a plurality of openings, which are optionally available for receiving the insertion sleeve and the biopsy element.

18. The biopsy system according to claim 1, wherein on the distal side the distal section of the fluorescence endoscopy element comprises an LED for the decoupling of stimulation light and/or on the distal side an image or a photodiode for the coupling in of fluorescence light.
